(19) United States
(12) Patent Application Publication
(10) Pub. No.: US 2008/0058761 A1
(43) Pub. Date: Mar. 6, 2008

(54) Diamond Dusted Extrusion Cannula

(76) Inventor: Richard Spaide, New York, NY (US)

Correspondence Address: HUGHES HUBBARD & REED LLP, ONE BATTERY PARK PLAZA, NEW YORK, NY, 10004, US

(21) Appl. No.: 11/679,692

(22) Filed: Feb. 27, 2007

Related U.S. Application Data
(60) Provisional application No. 60/778,672, filed on Mar. 2, 2006.

(51) Int. Cl. A61F 9/007; A61F 9/00; A61M 31/00

(52) U.S. Cl. 604/521; 606/161

(57) ABSTRACT

An aspirating cannula for ophthalmic surgery is provided, having a soft tip with an abrasive coating at its distal end. These features combine the functionality of a soft-tip abrasive instrument with means to remove fluid from the eye. The aspirating function of the cannula may be used to provide suction, thereby increasing the frictional force that can be applied by the abrasive tip coating, without requiring the surgeon to apply additional downward pressure on the retina. Several methods of advantageously using this cannula to perform ophthalmic surgical procedures are also disclosed.

### CROSS-REFERENCE TO RELATED APPLICATION

This application claims the benefit of the filing date of U.S. Provisional Application 60/778,672, filed on Mar. 2, 2006, entitled “Diamond Dusted Extrusion Cannula.”

### BACKGROUND OF THE INVENTION

1. Field of the Invention

The present invention is in the field of ophthalmic surgery, and more specifically concerns a cannula that combines aspirating and abrasive elements, and methods for its use.

2. Background of the Related Art

Surgical operations within the eye often involve scraping and removing membranes, blood clots and other materials from the eye, as well as removing marking agents and the like introduced in various examinations and treatments. Such operations often involve, or occur in connection with, the introduction or removal of fluids.

Removal of fluid and other material from the eye can be done by a number of means. An extrusion cannula is one instrument that has been used for this purpose, which uses suction (aspiration) to remove fluid through a tubular passageway internal to the cannula. However, use of an extrusion cannula or other instrument that is made of metal runs the risk of inadvertent impact between the instrument and delicate structures within the eye. For a number of years, ophthalmic surgical practice has involved the use of “soft-tip” cannulas, an example of which is shown in U.S. Pat. No. 6,800,076 by Humayun. Such a cannula may have an extrusion handle, a tubular metal body, which may be detachable from the handle by means such as a luer-lock, and a soft tip, at the far (distal) end of the metal body, comprising a short tubular section of a soft material such as silicon rubber or polyurethane.

Non-aspirating cannulas have also been developed for removing proliferative membranes within the eye. A tool for such a purpose, known as a “membrane eraser,” is shown in U.S. Pat. No. 5,921,998 to Tano, et al. This tool consists of a grip portion; a rod-shaped body attached to one end of the grip portion; an elastic body fitted along a direction toward a front end of the rod-shaped body to the front end side of the rod-shaped body; and hard inorganic fine-grains or particles fixed on a tapered tip of the elastic body. The ability of this instrument to engage epiretinal membranes is dependent on the friction created by dragging the instrument across the surface of the membrane. To increase the friction, thereby increasing the purchase on the membrane additional force needs to be placed on the instrument. Since delicate structures such as the retina, retinal pigment epithelium, and choroid are directly below the membrane, these structures may suffer damage.

Despite these developments, the field currently lacks a cannula that combines the ability to be used abrasively to remove membranes and other material with the ability to remove fluid from the eye.

### SUMMARY OF THE INVENTION

In one embodiment, the invention provides an aspirating cannula having a soft tip, the soft tip having an abrasive coating at the distal end, which provides the ability to use the instrument to remove membranes and the like, while also providing the ability, during the course of dislodging the membrane material, to remove the resulting debris by aspiration.

The aspirating cannula may be a disposable unit removably attached to an extrusion handle and used as an assembly with the extrusion handle.

The aspirating cannula may be attached to such a handle and used to remove matter, for example, proliferative membranes, abrasively from the surface of the retina, while in the same series of operations removing the dislodged debris by aspiration, thus overcoming the limitations of the prior art. Moreover, friction may be placed on the material by the abrasive nature of the coating. To increase the ability to engage material, the surgeon does not need to apply downward mechanical pressure. By increasing the suction through the cannula the membrane is brought into increasingly strong contact with the abrasive end of the cannula. This allows increased ability to remove material from the surface of intraocular structures without causing compressive damage.

The invention is further illustrated by the accompanying drawings and further explained in the detailed description that follows.

### BRIEF DESCRIPTION OF THE DRAWINGS

The figures are all schematic views in accordance with various embodiments of the invention.

FIG. 1 is a side (elevation) view of one embodiment of the invention, showing an assembly comprising an extrusion handle and a soft-tipped aspirating cannula.

FIG. 2 is an expanded side elevation of the distal portion of the cannula in FIG. 1.

FIG. 3 is an isometric (angled) view of the distal portion of an embodiment of a cannula in accordance with the invention, showing additional detail of its construction, and of the disposition of abrasive particles on the distal end.

### DETAILED DESCRIPTION

The following is a detailed description that illustrates some alternatives for making and using an aspirating cannula in accordance with this invention.

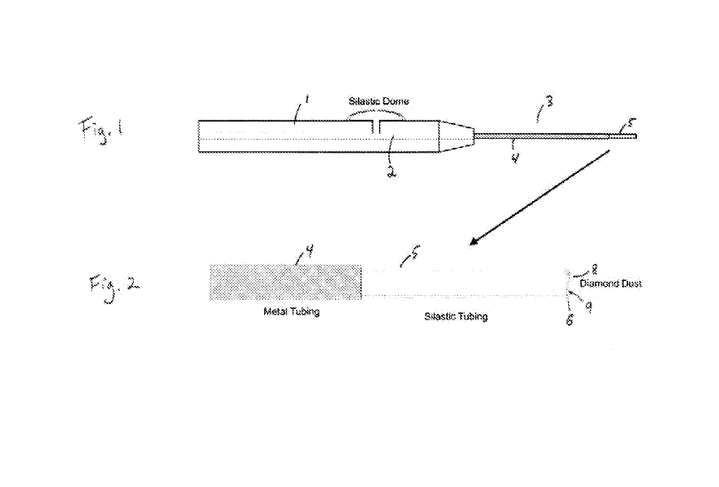

FIG. 1 shows a headpiece 1 having an internal passage 2, and a cannula 3 having a tubular body 4 and soft tip 5. In the embodiment shown, the tubular body and soft tip extend internal passage 2 to the distal end of the assembly. FIG. 1 shows that the tubular body 4 is attached to the headpiece 1. Preferably, the invention can be constructed as a detachable disposable cannula with a luer-lock designed to fit on an extrusion headpiece. It could also be frictionally attached. Additionally, the soft tip need not necessarily be tubular in shape; substantially flat, nontubular configurations would also be serviceable.

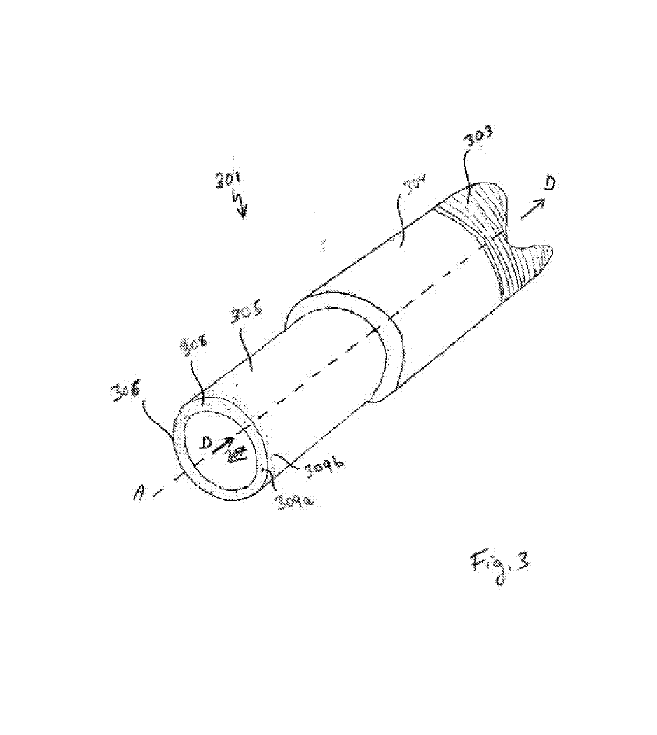

FIG. 2 is expanded view of the distal portion of the cannula in FIG. 1, showing that the tubular body 4 is metal, and that the soft tip 5 is an elastic body distally affixed to the tubular body and comprised of medical grade silicone elastomeric tubing. FIG. 2 further shows the elastic body ending at its distal end 6 a surface 8 having a flat cross-section substantially transverse to the longitudinal axis of the cannula. The distal surface 8 is coated with an attached layer 9 of abrasive particles. FIG. 3 is an isometric view of the distal portion of an alternative embodiment of a cannula in accordance with the invention, showing a one-piece unit with grip 303, tubular body 304, elastic body 305, distal surface 308, in the form of a flat face at distal end 306 substantially transverse to the longitudinal axis of the cannula, internal longitudinal passage 307 and abrasive particles 309a (on the face 308) and 309b (extending over onto the side of the distal end of the elastic body adjacent face 308).

Preferably, tubular body 4 is made of stainless steel, though other materials such as titanium may be used.

Using silicone tubing extending from the metal instrument offers the advantage of decreasing the likelihood of traumatic impact with delicate structures interior to the eye. Medical grade silicone elastomer is a preferred material, because in addition to its pliability, medical grade silicone elastomer has a relatively high chemical inertness. In alternative embodiments other materials may be used, such as polymers and elastomers, including but not limited to polyurethane elastomers, fluorosilicone, perfluoroelastomers, ethylene-vinyl acetate.

Friction is related to the coefficient of friction and force. The coefficient of friction is maximally increased in this embodiment by using abrasive material at the distal tip. Increasing the downward pressure could injure underlying tissues such as the retina, retinal pigment epithelium, or choroid. The medical grade silicone elastomeric tubing, being relatively flexible, deforms with pressure against intraocular structures. The flexibility of the tubing would ordinarily limit the ability of using the instrument to engage and abrade the surface of a structure. With the present embodiment the pressure applied on the tissue can be increased by either downward pressure or by increasing the suction within the tubing. Increasing suction would increase the frictional force without placing the underlying tissues such as the retina, retinal pigment epithelium, or choroid at risk for compressive injury.

By covering the end of the tubing with an abrasive such as diamond dust, the tubing is simultaneously flexible and also abrasive. This can be used to help dislodge blood clots from the surface of the retina, help remove triamcinolone injected into the eye as a marking agent, as well as to perform functions already done with typical fluid removal devices. In addition, the abrasive can be used to engage and remove unwanted membranes or structures within the eye. The instrument allows for the simultaneous benefits conferred by suction and abrasion. It is possible to grasp and slide sections of retina, such as in a retinal detachment secondary to a giant retinal tear. Optimization of the retinal reattachment procedure requires simultaneous ability to slide the retina atraumatically and the ability to remove subretinal fluid once the retina is in the correct position. Retinal attachments secondary to giant retinal tears are mobile and frequently the retina will retract or scroll posteriorly. Using the present invention allows stretching and unrolling of the retina anteriorly, with removal of membranes and excessive intraocular subretinal fluid at the same time. If the subretinal fluid is removed the surface tension will allow the retina to remain in place. By similar fashion macular holes can be made smaller during surgery by siding the perifoveal macular tissue centrally.

Preferably, the abrasive material used is diamond particles, as diamond has a relatively high chemical inertness and is hygienically an attractive material. In alternative embodiments other hard particles may be used, for example, marble, zirconium oxide, zirconia alumina, boron nitride, silicon dioxide, silicon nitride, silicon carbide, silica, alumina, crystal, quartz, sapphire, ruby, ceramic or the like. The size of the diamond (or other) particles is preferably in the diameter range of from approximately 2 μm to approximately 100 μm, and more preferably from approximately 10 μm to approximately 40 μm. The abrasive particles may be attached to the elastic body with a suitable adhesive; in the case of an elastic body made of medical grade silicone elastomeric tubing, the adhesive may be a silicon-based adhesive. It is possible to incorporate pharmaceutical agents, dyes, or marking agents in the coating to allow for localized application during use.

In a preferred embodiment, the distal end face of the cannula is substantially transverse with respect to the longitudinal axis of the assembly. This geometry permits a relatively smooth sweeping action to be performed on the surface of the retinal tissue to scrape off membrane matter and sweep up debris, marker material, etc. Furthermore, in this embodiment, the tubular internal passage within the cannula provides a ready means for removing the dislodged debris and other matter by aspiration.

In one method of using the invention, the cannula may be inserted onto the retina through a small incision. Using a sweeping action, membrane material may be abraded from the retinal surface with the diamond-dusted tip of the instrument, and the debris swept up and aspirated away with liquid through the internal longitudinal passage of the instrument (for example, see passage 307 and arrows “D” in FIG. 3). The instrument can also be used to blow out liquid to help move material on the retina.

Thus, the present invention permits the functions previously performed by a “membrane eraser” to be combined with the functions of an aspirating cannula, allowing unwanted material in the eye to be removed, collected and removed in a single operation without prolonged, repeated procedures, switching back and forth among differing instruments, and without unduly risking damage to delicate eye structures. Instead of relying on downward compressive force against the underlying membrane and retina the present invention allows for suction to increase the ability to engage and remove membranes, structures, and materials from the eye. The suction can also be used to move structures in the eye such as sliding the retina during retinal reattachment procedures. Fluid may be injected in through the cannula to clear unwanted material from the cannula, to disengage from intraocular structures, or to blow unwanted material from the surface of the retina. The cannula can be used to inject drugs, dyes, or marking agents into the eye through a sclerotomy opening.

The foregoing summary, drawings and detailed description describe various embodiments of the invention and the principles by which the invention operates, and show the advantages that the invention provides over previous solutions. It is believed that the invention has been explained in sufficient detail to enable persons of ordinary skill in this field who study this disclosure to practice the techniques shown, as well as other variations and embodiments within the spirit of the invention that suit their individual needs. Accordingly, the specific features described and shown herein to illustrate various aspects and embodiments of the invention are not intended to limit the scope of the invention. The scope of the invention should be understood as defined only by the claims set forth below.

I claim:

1. An aspirating cannula comprising a proximal end attachable to a handle, a tubular body extending from said proximal end and having a longitudinal internal passage therein, and a tip attached to the distal end of said tubular body, said tip comprising an elastic body made of soft material and having an abrasive coating on the distal end thereof.

2. An aspirating cannula in accordance with claim 1, wherein said soft material is medical grade silicone elastomer.

3. An aspirating cannula in accordance with claim 1, wherein said abrasive coating comprises a plurality of hard, fine-grained particles fixed on said elastic body.

4. An aspirating cannula in accordance with claim 3, wherein said fine-grained particles are diamond particles.

5. An aspirating cannula in accordance with claim 3, wherein said fine-grained particles are selected from the group consisting essentially of marble, zirconium oxide, zirconia alumina, boron nitride, silicon dioxide, silicon nitride, silicon carbide, silica, alumina, crystal, quartz, sapphire, ruby and ceramic particles.

6. An aspirating cannula in accordance with claim 3, wherein said fine-grained particles are fixed to said elastic body by a silicone-based adhesive.

7. An aspirating cannula in accordance with claim 3, wherein said fine-grained particles comprise grains having a range in diameter from about 2 μm to about 100 μm.

8. An aspirating cannula in accordance with claim 7, wherein said range is further limited to a range of from about 10 μm to about 40 μm.

9. An aspirating cannula in accordance with claim 1, wherein said tubular body is made of metal.

10. An aspirating cannula in accordance with claim 9, wherein said metal is stainless steel.

11. An aspirating cannula in accordance with claim 9, wherein said metal is titanium.

12. An aspirating cannula in accordance with claim 1, wherein said proximal end is attachable to a handle by a friction attachment.

13. An aspirating cannula in accordance with claim 1, wherein said proximal end is attachable to a handle by a luer-lock.

14. An aspirating cannula in accordance with claim 3, wherein said elastic body is tubular and extends said longitudinal internal passage of said tubular body, the distal end of said elastic body has a face that is substantially transverse to the longitudinal axis of the tubular body and elastic body, and at least a portion of said fine-grained particles are fixed to said face.

15. An aspirating cannula in accordance with claim 14, wherein a portion of said fine-grained particles are also fixed to the outer surface of said elastic body adjacent said face, thereby forming an abrasive-coated tip on said elastic body.

16. An extrusion cannula comprising an extrusion handle and an aspirating cannula in accordance with claim 1.

17. An extrusion cannula comprising an extrusion handle and an aspirating cannula in accordance with claim 11.

18. A method for performing ophthalmologic surgery, comprising making a sclerotomy incision in the eye, inserting through said incision an aspirating cannula constructed in accordance with claim 1, using the abrasive distal end of said cannula to apply frictional force to structures within the eye, and removing fluid from the eye by aspiration through the longitudinal internal passage of said aspirating cannula.

19. A method in accordance with claim 18, for removing matter from the surface of the retina, further comprising sweeping said surface with the abrasive distal end of said cannula, so as to dislodge said matter, and removing said matter by aspiration through the longitudinal internal passage of said aspirating cannula.

20. A method in accordance with claim 18, further comprising using the suction created by removing fluid from the eye through said cannula to increase the friction provided by said abrasive distal end thereof, without increasing the downward force on said cannula.

21. A method in accordance with claim 20, further comprising using said frictional force, combined with and amplified by said suction, to grasp and slide sections of detached retinal tissue.

22. A method in accordance with claim 20, further comprising using said frictional force, combined with and amplified by said suction, to move structures in the eye during retinal reattachment procedures, and to remove subretinal fluid so as to promote retention of the retina by surface tension.

23. A method in accordance with claim 20, further comprising using said frictional force, combined with and amplified by said suction, and siding the perifoveal macular tissue centrally to reduce the size of macular holes.

24. A method for performing ophthalmologic surgery, comprising making a sclerotomy incision in the eye, inserting through said incision an aspirating cannula constructed in accordance with claim 1, and injecting fluid through said cannula.

25. A method in accordance with claim 24, wherein said fluid is injected to clear unwanted material from the cannula.

26. A method in accordance with claim 24, wherein said fluid is injected to disengage from intraocular structures.

27. A method in accordance with claim 24, wherein said fluid is injected to blow unwanted material from the surface of the retina.

28. A method in accordance with claim 24, wherein said fluid comprises a drug, a dye, or a marking agent.
